(19) United States
(12) Patent Application Publication
(10) Pub. No.: US 2007/0023309 A1
(43) Pub. Date: Feb. 1, 2007

(54) Sterilization pouch for medical instruments and methods of use

(75) Inventor: Phillip Davis, Waxhaw, NC (US)

Correspondence Address: EDWARDS & ANGELL, LLP, P.O. BOX 55874, BOSTON, MA, 02205, US

(73) Assignee: General Hospital Supply Corporation

(21) Appl. No.: 11/190,442

(22) Filed: Jul. 27, 2005

(51) Int. Cl. B65D 83/10; A61B 19/02

(52) U.S. Cl. 206/438; 206/363

(57) ABSTRACT

A pouch is disclosed for protecting delicate medical instruments during ethylene oxide and steam sterilization procedures, which is formed by opposed front and rear panels, wherein one of the panels has a flap portion for enclosing the pouch when a medical instrument is placed inside for sterilization.

### BACKGROUND OF THE INVENTION

1. Field of the Invention

The present invention relates to an instrument holder and method of sterilization of medical instruments. More particularly, the present invention relates to a pouch for insertion and retention of medical instruments and a method of sterilization of the instruments therein using steam and/or ethylene oxide.

2. Description of the Related Art

Various packaging and containers into which small medical instruments such as clamps, scissors, retractors, pliers, and the like, are placed during steam and/or ethylene oxide (EO) sterilization are known. Currently in the field, paper and plastic pouches, called peel pouches are widely used. These rectangular pouches, such as Converters® Paper Tubing (Cat. T97006) available from Cardinal Health, McGaw Park, Ill., have paper on one side and clear plastic on the other allowing view of items once inserted therein. Instruments are inserted by one end into the narrow top opening of the peel pouch, and down into the length of pouch. After sterilization, the pouch is opened by pulling apart the paper and plastic sides.

Additionally in the art are instrument holders that provide an open-ended pocket or plurality of pockets with each individual pocket receiving an instrument, in order to protect instrument tips from puncturing the holder or injuring a user or from becoming damaged or dulled themselves. See U.S. Pat. No. 4,385,692 to Eldridge, Jr.; U.S. Pat. No. 4,342,390 to Mitchell et al, Additionally, instrument pouches with this arrangement in the field of H₂O₂ gas plasma sterilization are likewise known. See U.S. Pat. No. 6,391,260 to Davis et al.

These existing instrument holders and pocket organizers are problematic, specifically when inserting instruments and items into the small opening of the various pockets of the holder or at one end of the vertical length of the peel pouch. Instruments tend to stick within these long holders and pouches upon insertion and removal, and this becomes particularly problematic when it comes time to quickly remove them for use through the narrow opening at one end. As such, instruments cannot be accessed quickly and/or may become contaminated in the struggle to remove them.

In addition to insertion and removal impediments, peel pouches currently available in the art have the additional problems of inadequate sterilization when used within wrapped sets or containers as the pouches cannot be positioned to ensure adequate air removal, steam contact, and drying. See The Association for Advancement of Medical Instruments (ANSI/AAMI), ST46, p. 31, 5.6.2.4 Paper/plastic Pouches (2002). Clearly there remains a need in the art for pouches appropriate for use with wrapped sets or containers and/or moist heat (steam) or ethylene oxide (EO) sterilization of medical instruments.

### SUMMARY OF THE INVENTION

In one aspect of the present invention, an instrument holder comprising at least one pouch having a first layer, a second layer defining an opening and a flap extending from either the first or second layer and receiving at least one instrument laterally into the opening of the pouch or pouches, the flap folding over to close the pouch, is provided.

In one aspect of the present invention, a sterilizing set for holding instruments to be sterilized comprising at least one pouch, each pouch having a first layer and a second layer defining an opening for receiving at least one instrument inserted laterally into the opening of the pouch, and a flap extending from either the first or second layer, the flap folding over to close the pouch; and a sterilization wrap to cover, enclose or wrap the pouch or pouches during sterilization, is provided. In another aspect of the present invention, a method of sterilizing instruments comprising at least one pouch, each pouch having a first layer and a second layer defining an opening and a flap extending from either the first or second layer; inserting laterally at least one instrument into the opening of the pouch or pouches; folding the flap over the opening of each pouch to close the instrument or instruments in the pouch or pouches; and placing the pouch or pouches into a sterilization unit, is provided.

The subject invention is also directed to a pouch for protecting delicate medical instruments during sterilization and transport to an operating room. This pouch, which is particularly well adapted for use during ethylene oxide and steam sterilization procedures, includes a generally rectangular envelope-like structure formed by opposed front and rear panels that are attached to one another along bottom and side edges. The rear panel of the envelope-like structure has a generally trapezoidal flap portion that extends above a top edge of the front panel to enclose the pouch when a medical instrument is placed inside for sterilization and/or transport. Preferably, the pouch is formed from a firm hydrophobic foam material. It is envisioned that this pouch can have a broad range of sizes, but in one particular instance, the envelope-like structure has a length of about 12″ and a depth of about 5″ so that it is dimensioned to hold a fairly broad range of medical instruments.

These and other aspects of the instrument protector pouch and methods of using the same will become more readily apparent to those having ordinary skill in the art from the following description of the invention taken in conjunction with the drawings described hereinbelow

### BRIEF DESCRIPTION OF THE DRAWINGS

So that those having ordinary skill in the art to which the subject invention pertains will more readily understand how to make and use the subject invention, preferred embodiments thereof will be described in detail hereinbelow with reference to the drawings, wherein:

FIG. 1 is a perspective view of one embodiment of the present invention, showing a generally triangular shaped sterilization pouch in open position;

FIG. 2 is a perspective view of the embodiment of FIG. 1, showing the sterilization pouch in closed position;

FIG. 3 is a perspective view of another embodiment of the present invention, showing the envelope-like sterilization pouch in open position;

FIG. 4 is perspective view of the embodiment of FIG. 3, showing the envelope-like sterilization pouch in closed position, with flap folded outside the pouch; and

FIG. 5 is a perspective view of the embodiment of FIG. 3, showing sterilization pouch in closed position with flap folded inside the pouch.

### DETAILED DESCRIPTION OF THE PREFERRED EMBODIMENTS

According to the present invention, a method of retaining instruments during sterilization process and additionally, for storing and protecting instruments afterwards so they remain sterile until needed for use is provided as an instrument holder comprising at least one pouch. In one aspect of the present invention, the pouch has an edge or length of the pouch, which is greater than at least two other lengths defining an opening for lateral insertion of at least one instrument.

In another aspect of the present invention, a method of steam or ethylene oxide sterilization is provided where the instrument pouch receives at least one instrument. In another aspect of the present invention, a sterilization set comprising at least one pouch and sterilization wrap to cover, enclose or wrap each pouch singly and/or wrap each pouch together, is provided. In another aspect of the present invention, a method of sterilization is provided using at least one pouch contained in a wrapped set, tray or container, which is placed in the sterilization unit.

Pouch and any and all components of the pouch or instrument holder according to the present invention, such as flap and layers, may be made of any suitable material appropriate for use in a sterilization process. The material may be penetrated by sterilant to achieve adequate sterilization of instrument or instruments therein and later provide adequate air removal and drying instrument or instruments therein after sterilization. Suitable materials may include foam, plastic, paper, and any other materials that may be flexible, reticulated, perforated, porous, and/or cellular. Preferably, the pouch and any and all components thereof are at least partially made of foam. An example of foam preferentially employed in the present invention is molded urethane foam available from the Wm T. Burnett & Co. Foam Division, Jessup, Md. (Polyester Polyurethane Foam No. S82N). The pouch and any and all components of the pouch or instrument holder according to the present invention may be formed by any appropriate means including but not limited to techniques of molding, cutting, heat sealing, and/or radio frequency (RF) welding.

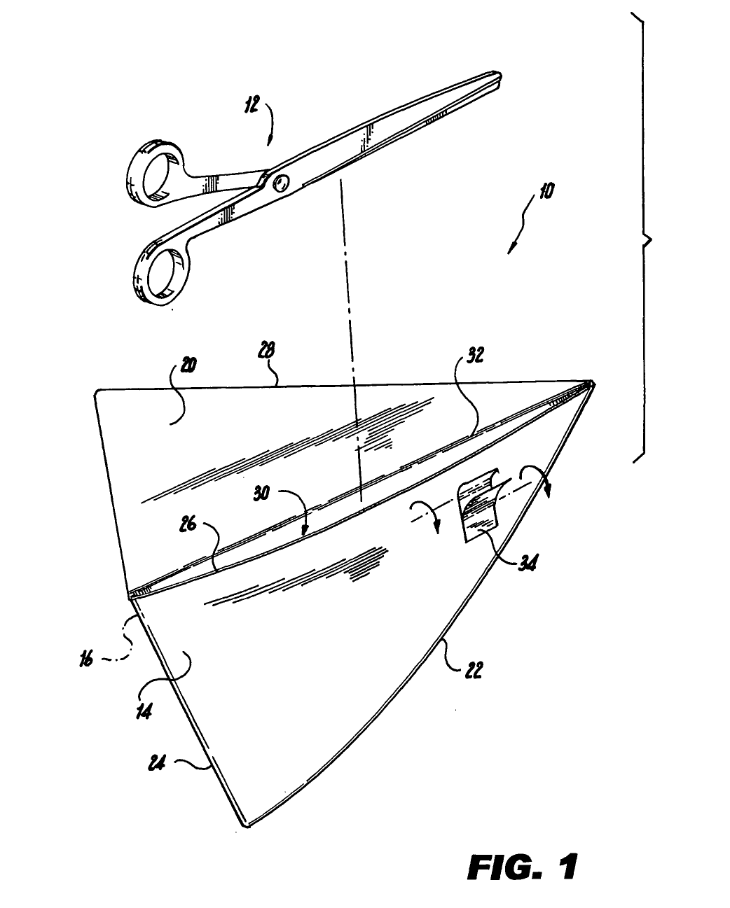

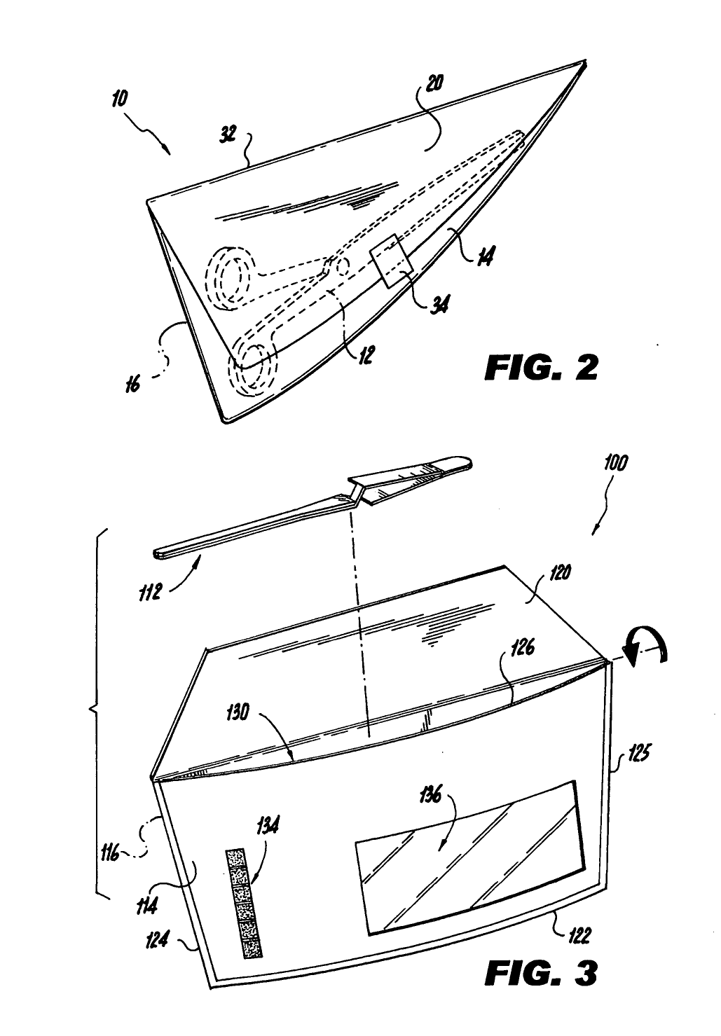

Referring to FIG. 1, a sterilization pouch or instrument holder according to the present invention is shown generally at 10, in open position ready for receipt of instrument 12. Pouch 10 has two layers 14, 16 of material, preferably at least partially made of foam, and more preferably, entirely made of foam. Pouch 10 has at least two edges, shown in FIG. 2 specifically with three edges 22, 24, 26. Layers 14, 16 are sealed together along at least one edge, shown here sealed at two edges, bottom edge 22 and side edge 24. Layers are not sealed at top edge 26 which is also the free edge of the first layer 14. Top edge 26 and second layer 16 together define the opening 30 of pouch 10 that is capable of receiving at least one instrument 12 when in open position. Extending upward from the second layer 16 is flap 20.

In one aspect of the present invention, opening 30 is disposed on edge 26 of pouch that defines the longest length of pouch 10. As shown in FIG. 1, top edge 26 defining opening 30 is longer than other edges 22, 24. Opening 30 disposed at the long length of the pouch 10 allows for lengthwise insertion (and later removal) of instruments which is easier and less time consuming, as instruments can be placed in and out the large opening 30 rather than forced or squeezed into a small opening tip-first into an instrument holder.

Referring to FIG. 3, in another aspect of the present invention, pouch may have four edges 122, 124, 125, 126. Layers 114, 116 are sealed together along three edges, specifically shown as edge 122 at bottom of pouch 10 and edges 124, 125 at side of pouch 100. Layers are not sealed at top edge 126, which is defined by the free edge of the first layer 114 of material.

Additionally, adjacent to top edge 126, flap 120 extends upward from second layer 116. Top edge 126 and second layer 116 together define the opening 130 of pouch 100 that is capable of receiving at least one instrument 112 when in open position. Extending upward from the second layer 116 is flap 120. In one aspect of the present invention, the opening 130 is disposed on the edge 126 of pouch that is longer than at least two other edges, and further may be longer than all other edges, as shown in FIG. 3, as edge 126 is longer than side edges 124, 125 and bottom edge 122. As previously discussed, an opening along the long edge of the pouch allows for lateral insertion, and hence, ease of insertion of instruments in the pouch to be sterilized, and later, ease of removal of sterilized instruments therefrom.

Referring again to FIG. 1, in one aspect of the present invention, pouch 10 may have a weld 32 disposed in between the second layer 16 and flap 20 to facilitate bending of the material along the weld 32 when placing pouch 10 in closed position. FIG. 2 shows pouch 10 in closed position, with flap 20 folded at weld 32 to meet opposite layer from which it extends, specifically shown as first layer 14, to enclose the at least one instrument 12 in the pouch 10. Alternatively, as shown in FIG. 3, flap 120 may be a continuous piece of material extending from second layer 116 of pouch 100 and be made of a suitably material, preferably foam, so that it may easily be bent in order to close opening 130. FIG. 4 shows pouch 100 in closed position, with flap folded over to meet the opposite layer from which it extends, specifically the first layer 114. Alternatively, the flap may be “tucked in” or folded over into the inside of the pouch in order to place the pouch in a closed position.

Flap 120 is adhered to first layer 114 by use of a suitable closure or attachment means, such as tape 140, as shown in FIG. 3, adhesive, tab, clip or the like. Alternatively, a layer of the pouch may be modified to receive the tab and the flap in closed position. In one example, first layer may have a slit or multiple slits disposed therein, into which the tip of flap is inserted to hold it in closed position. In another example, a strip or strips of suitable material, preferably foam, may be disposed on first layer so that tip of flap may be inserted between strip or strips and the first layer to hold flap in closed position.

In another aspect of the present invention, sterilization indicator may be disposed on the pouch to indicate when sterilization is complete, typically by a color change. Referring to FIGS. 1-2, sterilization indicator 34 may be releasably adhered to a surface of the pouch, such as the flap 20, first 14 layer or second 16 layer, shown in FIG. 1 prior to closure of pouch 10, on first layer 14. Once flap 20 is closed over sterilization pouch or set 10, as shown in FIG. 2, sterilization indicator 34 may be removed from pouch 20 and re-adhered over flap 20 to ensure its closure during and after sterilization until instruments are to be removed for use. Sterilization indicator 34 may dually serve as both an indicator of completion of sterility and as a closure means for the pouch.

Referring to FIGS. 3-4, in another aspect of the present invention, sterilization indicator 134 may be fixedly disposed on or integrated into the pouch 100 in a location visible when sterilization pouch or set 100 is in closed position, shown in FIGS. 3-4 disposed on first layer 114, to indicate completion of sterilization, for instance, by a color change. Sterilization indicator 134 may be made of any appropriate material suitable for use in sterilization process, such as foam or paper. Sterilization indicator 134 may be made of the same or different material than the pouch 100, and integrated or inserted into the material of the pouch 100 or applied thereon after manufacture of the pouch. Additionally, tape 140 used to close flap 120 may be specialized to have the additional ability to indicate completion of sterilization, such as by color change, and in this regard serve a dual purpose, making the need for a separate sterilization indicator 134 unnecessary.

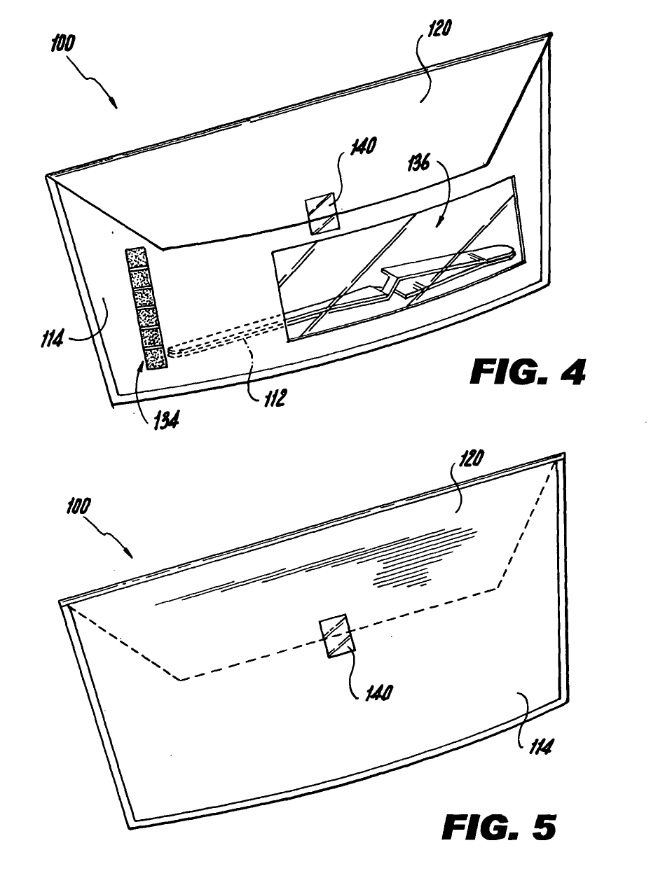

Alternatively, as shown in FIG. 5, flap 120 may be “tucked in” or folded over into the inside of the pouch 100 in order to place the pouch in a closed position. Optionally, once in this position, tape 140 may be adhered to the flap 120 and portion of the pouch 100, shown in FIG. 5 adhered to first layer 114, to retain pouch 100 in a closed position.

In another aspect of the present invention, sterilization indicator may be separate from the pouch and disposed on or within the tray or closed container. Sterilization indicator disposed on or within the tray or container may be in addition to sterilization indicator 134 disposed on the pouch.

The sterilization pouch of the present invention is preferably made at least partially, and more preferably entirely of foam, or a similarly sterilant-penetratable and dryable material that will ensures proper sterilization, by providing adequate sterilant (such as steam) contact, during sterilization and air removal and drying after sterilization (by steam or ethylene oxide sterilization). Preferably, the pouch is formed from a firm hydrophobic foam material. The use of foam or other like material to construct the pouch additionally protectively cushions instruments and prevent them from bouncing around when they are placed in wrapped sets or containers for sterilization.

Referring to FIGS. 3-4 in one aspect of the present invention, pouch 100 may be at least partially made of an at least partially translucent material 136, such as translucent foam or plastic, to reveal the contents of the pouch when closed. Use of an at least partially translucent material allows for identification of the instrument contained in a closed pouch and ease of selection of the appropriate sterilized instrument by the user.

Referring to FIGS. 3-4, in another aspect of the present invention, one layer, such as first layer 14 as shown in FIG. 1 may be made of an at least partially translucent material, such as plastic or translucent foam, and a second layer, such as second layer 16, may be made of material that is non-translucent. Preferably, foam material itself may be at least partially translucent to allow user to view its content, removing the need for constructing a pouch of two separate materials.

Preferably, the pouch of the present invention has four edges (quadrilateral), with the opening disposed on the edge that is longer or greater in dimension than at least two of the other three edges. In one example, pouch may be substantially rectangular in shape, flap extending from one rectangular layer, noting the “top” of the pouch. In this example, two side edges of substantially equal length would measure approximately two inches to approximately six inches, and top and bottom edges with substantially equal lengths would measure five to twenty-eight inches, with opening disposed along the top edge, which is longer or greater in dimension than the two sides. This results in various possible pouch sizes, in height by length, including but not limited to: 3″ by 5″; 2″ by 20″; 5″ by 9½″; and 6″ by 28″.

While it is envisioned that the envelope-like rectangular pouch can have a broad range of sizes, in one particular instance, the structure has a length of about 12″ and a depth of about 5″ so that is dimensioned to hold a fairly broad range of medical instruments.

In another example, the pouch may be substantially trapezoidal in shape, with the opening disposed along the edge longer than the other three edges. In this example, the dimensions would change for the bottom edge, resulting in various possible pouch sizes, in height by top length and bottom length, including but not limited to, 3″ by 5″ top and 1½″ bottom; 2″ by 20″ top and 14″ bottom; 5″×9½″ top and 4″ bottom; and 6″ by 28″ top and 20″ bottom.

In one aspect of the present invention, at least one pouch is placed in sterilization tray or basket as part of a wrapped set to be placed in sterilization unit for sterilization. Sterilization wrap, such as KIMGUARD, commercially available from Kimberly-Clark Health Care, Roswell, Ga., is placed over and/or around the tray to enclose the at least one pouch therein. In another aspect of the present invention, at least one pouch may be placed in a container, which is closed with a lid when placed in sterilization unit. In another aspect of the present invention, a sterilization set includes at least one pouch with an opening in its long dimension side for receipt of at least one instrument to be sterilized, and a sterilization wrap to enclose the at least one pouch before placement in the sterilization unit.

In another aspect of the present invention, pouch may be used to hold a count sheet. Depending on size of pouch used, user may have to first fold count sheet, place it in pouch, fold over flap and tape shut for placement in the sterilization unit. The term “sterilization unit” refers to any machine or apparatus utilizing any process, including but not limited to steam, gas, and plasma, for sterilizing and decontaminating medical instruments according to the present invention, currently known and which may be developed in the future.

The present invention overcomes problems in the art related to inadequate steam penetration of the traditional paper and plastic pouches in wrapped sets or containers and difficulty of vertical insertion of instruments into sterilization pouches into a shorter edge of the pouch. The present invention provides a low profile and light-weight instrument holder and pouch achieving exceptional sterilization results. Further, preferable construction of the pouch with foam or other like material, cushions and protects instruments inserted in the pouch and provides a sterile storage means as instruments may be left in the pouch until time for use.

The pouch according to the present invention may be used in wrapped sets, closed containers or wrapped individually in sterilization wrap for placement in a sterilization unit. The present invention additionally provides a method of sterilization using the pouch in closed containers, and wrapped sets, or providing pouch alone or pouches wrapped in sterilization wrap.

The above description is for the purposes of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence, which is effective to meet the objective there intended, unless the context specifically indicates to the contrary.

1. An instrument holder, comprising:
a) at least one pouch having:
ii) a first layer with at least first and second edges,
ii) a second layer with at least first and second edges, with at least the first edge of the first layer sealed to the first edge of the second layer and the second edges of the first and second layers defining an opening for insertion of at least one elongate instrument laterally into the opening, and
iii) a flap extending from either the first or second layer, the flap capable of folding over the opening to place the pouch in a closed position.

2. The instrument holder of claim 1, further comprising sterilization wrap to wrap the at least one pouch prior to placement in a sterilization unit.

3. The instrument holder of claim 1, wherein the first and second layers are formed from a same piece of material and wherein at least one edge of the pouch is a fold.

4. The instrument holder of claim 1, wherein the pouch is at least partially made of a foam material.

5. The instrument holder of claim 1, wherein the pouch is at least partially made of an at least partially translucent material to reveal the contents of the pouch when closed.

6. The instrument holder of claim 1, further comprising at least one sterilization indicator.

7. The instrument holder of claim 1, wherein the first layer and the second layer of the at least one pouch each have at least three edges, with the third edge of the first layer sealed to the third edge of the second layer, and wherein the second edges defining the opening of the pouch are longer in dimension than at least the first edges and the third edges of each layer to allow insertion of the at least one instrument laterally into the opening at the long dimension edge of the pouch.

8. The instrument holder of claim 1, further comprising a container to hold the at least one pouch and a lid to close the container prior to placement in a sterilization unit.

9. The instrument holder of claim 1, further comprising a sterilization tray to hold the at least one pouch and sterilization wrap to cover the tray prior to placement in a sterilization unit.

10. The instrument holder of claim 1, wherein the layer from which the flap extends has a weld disposed therein where the flap folds over to close the pouch.

11. The instrument holder of claim 1, wherein the flap is formed from a continuous piece of material with the layer from which it extends.

12. The instrument holder of claim 1, wherein the flap is held by tape to the opposite layer of the pouch from which it extends when closed.

13. The instrument holder of claim 1, wherein the flap is folded into the interior of the pouch.

14. The instrument holder of claim 1, wherein the flap is folded outside the pouch.

15. The instrument holder of claim 1, further comprising a plurality of pouches, wherein each of the plurality of pouches is configured and adapted to receive at least one elongate instrument laterally through the opening of each pouch and wherein the opening of each of the pouches is closed by folding the flap over the opening of each of the pouches.

16. A sterilization set for holding instruments to be sterilized, comprising:
a) at least one pouch having a first layer with at least first and second edges, a second layer with at least first and second edges, with at least the first edge of the first layer sealed to the first edge of the second layer and the second edges of the first and second layers defining an opening, and a flap extending from either the first or second layer;
b) at least one elongate instrument inserted laterally into the opening of the pouch and closed in the pouch by folding the flap over the opening of the pouch; and
c) a sterilization wrap to at least cover the at least one pouch prior to placement in a sterilization unit.

17. The sterilization set of claim 16, wherein the first and second layers are formed from a same piece of material and wherein at least one edge of the at least one pouch is a fold.

18. The sterilization set of claim 16, wherein the at least one pouch is at least partially made of a foam material.

19. The sterilization set of claim 16, wherein the at least one pouch is at least partially made of an at least partially translucent material to reveal the contents of the pouch when closed.

20. The sterilization set of claim 16, further comprising at least one sterilization indicator placed within the set.

21. The sterilization set of claim 16, wherein the first layer and the second layer of the at least one pouch each have at least three edges, with the third edge of the first layer sealed to the third edge of the second layer, and wherein the second edges defining the opening of the at least one pouch are longer in dimension than at least the first edges and the third edges of each layer to allow insertion of the at least one instrument laterally into the opening at the long dimension edge of the pouch.

22. The sterilization set of claim 16, wherein the first and second layer each have a top, a bottom, and two side edges, with the bottom and side edges of the first and second layers sealed, defining a substantially rectangular pouch with the top edges defining an opening longer in dimension than at least the two side edges to allow insertion of the at least one instrument into the opening at the laterally when the pouch is in open.

23. The sterilization set of claim 16, wherein the layer from which the flap extends has a weld disposed therein where the flap folds over to close the pouch.

24. The sterilization set of claim 16, wherein the flap is made of a continuous piece of material with the layer from which it extends.

25. The sterilization set of claim 16, wherein the flap is held by tape to the opposite layer of the pouch from which it extends when closed.

26. The sterilization set of claim 16, wherein the flap is folded into the interior of the pouch.

27. The sterilization set of claim 16, wherein the flap is folded outside the pouch.

28. The sterilization set of claim 16, further comprising at least one sterilization indicator.

29. The sterilization set of claim 16, wherein the sterilization wrap wraps around the at least one pouch prior to placement in the sterilization unit.

30. The sterilization set of claim 16, further comprising a plurality of pouches, wherein at least one elongate instrument is inserted laterally into the opening of each of the plurality of pouches and closed in each of the pouches by folding the flap over the opening of each of the pouches and wherein the sterilization wrap wraps around the plurality of pouches prior to placement in the sterilization unit.

31. The sterilization set of claim 16, further comprising a sterilization tray to hold the at least one pouch and wherein the sterilization wrap covers the tray and the at least one pouch therein prior to placement in the sterilization unit.

32. A method of sterilizing instruments, comprising:
a) providing at least one pouch made at least partially of foam, comprising:
i) a first layer with at least first and second edges,
ii) a second layer with at least first and second edges, with at least the first edge of the first layer sealed to the first edge of the second layer and the second edges of the first and second layers defining an opening, and
iii) a flap extending from either the first or second layer;
b) inserting laterally at least one elongate instrument into the opening of the at least one pouch;
c) folding the flap over the opening of the at least one pouch to close the instruments in the pouch; and
d) placing the at least one pouch into a sterilization unit.

33. The method of claim 32, wherein the sterilization unit sterilizes instruments using steam or ethylene oxide.

34. The method of claim 32, further comprising wrapping the at least one pouch with sterilization wrap prior to placing into the sterilization unit.

35. The method of claim 32, further comprising placing the at least one pouch into a sterilization tray and covering the sterilization tray and the at least one pouch therein with sterilization wrap prior to placing the tray into the sterilization unit.

36. The method of claim 32, further comprising placing the at least one pouch into a container and placing a lid on the container to close the container prior to placing the closed container in the sterilization unit.

37. The method of claim 32, wherein the first and second layers are formed from a same material and wherein at least one edge of the pouch is a fold.

38. The method of claim 32, wherein the pouch is at least partially made of a foam material.

39. The method of claim 32, wherein the first or second layer of the pouch is at least partially made of an at least partially translucent material to reveal the contents of the pouch when closed.

40. The method of claim 32, wherein the first layer and the second layer each have at least three edges, with the third edge of the first layer sealed to the third edge of the second layer, and wherein the second edges defining the opening of the pouch are longer in dimension than at least the first edges and the third edges of each layer.

41. The method of claim 32, wherein the first and second layer each have a top, a bottom, and two side edges, with the bottom and side edges of the first and second layers sealed, defining a substantially rectangular pouch with the top edges defining an opening greater in dimension than at least the two side edges into which instruments are inserted laterally when the pouch is in open.

42. The method of claim 32, further comprising taping the flap closed to the opposite layer of the pouch from which it extends after placing the at least one instrument therein.

43. The method of claim 32, further comprising disposing at least one sterilization indicator with the at least one pouch prior to placing in the sterilization unit.

44. The method of claim 32, further comprising providing a plurality of pouches, wherein at least one instrument is inserted laterally into the opening of each of the plurality of pouches and closed in each of the pouches by folding the flap over the opening of each of the pouches.

45. A pouch for protecting medical instruments during sterilization comprising:
a generally rectangular envelope-like structure formed by opposed front and rear panels that are attached to one another along bottom and side edges, the rear panel of the structure having a generally trapezoidal flap portion that extends above a top edge of the front panel for enclosing the pouch when a medical instrument is placed inside for sterilization, and wherein the pouch is formed from a firm hydrophobic foam material.

46. A pouch as recited in claim 45, wherein the envelope-like structure has a length of about 12″ and a depth of about 5″.

47. A pouch as recited in claim 45, wherein the envelope-like structure is adapted for use in ethylene oxide and steam sterilization.
